(19) United States
(12) Patent Application Publication
(10) Pub. No.: US 2004/0142944 A1
(43) Pub. Date: Jul. 22, 2004

(54) Compositions for nasal application

(76) Inventors: Peter Serno, Bergisch Gladbach (DE); Andreas Ohm, Neuss (DE); Wolfgang Barth, Wuppertal (DE); Richard-Josef Bauer, Erkrath (DE); Hans-Martin Siefert, Wuppertal (DE); Dietmar Zimmer, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/473,000

(22) PCT Filed: Apr. 10, 2002
(86) PCT No.: PCT/EP02/03977

(30) Foreign Application Priority Data
Apr. 12, 2001 (DE) 101 18 305.4

(51) Int. Cl. A61K 31/519

(52) U.S. Cl. 514/262.1; 514/252.16

(57) ABSTRACT

The invention relates to compositions for nasal application containing cGMP-PDE inhibitors, especially PDE5 inhibitors, also containing a small amount of local anaesthetic in addition to said cGMP PDE inhibitor.

The present invention relates to compositions of cGMP PDE inhibitors, especially of PDE5 inhibitors, for nasal administration which, besides the cGMP PDE inhibitor, contain a small amount of a local anaesthetic.

Cyclic guanosine-3′, 5′-monophosphate phosphodiesterase inhibitors, abbreviated to cGMP PDE inhibitors, have a well known range of effects (cf., for example, EP-A-0 463 756, WO 99/24433). Inter alia, the biochemical bases of the process of penile erection were elucidated a few years ago and, on this basis, it was reported that cGMP PDE inhibitors, in particular PDE5 inhibitors, are suitable for treating male erectile dysfunction (cf. Rajfer et al., New England J. Med. 326 (1992), 90; Murray, Drug News & Perspectives 6 (1993), 150). Subsequently, the use of certain cGMP PDE inhibitors for treating male erectile dysfunction was described in WO 94/28902, and one of these (sildenafil citrate, Viagra®) is now proved as medicament which can be administered orally for this indication. One disadvantage of oral administration is, however, that the onset of action is delayed, which is deleterious to the spontaneity desired by the patient especially in this indication. In addition, first pass effects or food effects may impair the efficacy of an orally administered medicament.

In principle, it ought to be possible by nasal administration of an active ingredient to achieve a faster rise in the level of active ingredient in the blood stream and, associated therewith, an accelerated onset of action. There has thus been no lack of proposals in the prior art that cGMP PDE inhibitors be administered nasally, especially for treating male erectile dysfunction (cf. WO 96/32003, WO 97/03985, WO 98/53819, WO 99/24433, EP-A-0 967 214, WO 00/00199). For example, EP-A-0 967 214 describes nasal administration of a sildenafil salt which has better solubility in water, namely sildenafil mesylate, and the faster rise in the level of active ingredient in the blood stream which can be achieved thereby with a smaller amount of active ingredient being necessary compared with the oral route.

However, problems may arise on nasal administration of cGMP PDE inhibitors. Owing to their mechanism of action, these substances are vasodilators. Since PDE5 also occurs in the tissue of the nasal cavity, nasal administration of PDE 5 inhibitors leads to local dilation of the vessels of the nasal mucosa. The result is a condition in the nose which the patient finds unpleasant, such as itching or stinging, or eye-watering, an increase in the nasal airway resistance and/or a nasal blockage, although no local irritation is detectable toxicologically. Although it was described in EP-A-0 967 214 that these effects do not impair rapid absorption of sildenafil mesylate, the unpleasant condition in the nose, which is found to be upsetting particularly during sexual intercourse, the increase in the nasal airway resistance or the nasal blockage remain a not inconsiderable disadvantage.

EP-A-0 992 240, which corresponds to WO 98/53819, proposes to avoid an inadequate absorption of the cGMP PDE inhibitor, caused by the abovementioned disadvantages, by adding vasoconstricting active ingredients such as epinephrine, naphazoline nitrate, tramazoline hydrochloride or tetrazoline, antiallergic substances such as sodium cromoglicate or ketotifen fumarate, suppressors of nasal mucosal secretion such as flutropium bromide or steroids such as, for example, prednisolone, without showing by way of example that this sufficiently prevents the occurrence of the unpleasant feeling for the patient which has been described above.

Nasal administration of local anaesthetics has to date been disclosed for surface anaesthesia before surgical operations in the nasal region. In addition, U.S. Pat. No. 4,602,099 has described the use of local anaesthetics as adjuvants in antirhinoviral medicaments for additional treatments of the symptoms of an antirhinovirus infection. The only example of a local anaesthetic used in this patent was benzyl alcohol. It should be noted that benzyl alcohol is also known as preservative or as solubilizer and is described in these functions in EP-A-0 967 214 and WO 00/00199 as one of a plurality of adjuvants which can be used additionally for the formulations mentioned therein. In addition, it has emerged within the scope of the present invention that benzyl alcohol is unable to reduce or prevent the disadvantages described above which occur on nasal administration of cGMP PDE-inhibitors.

WO 99/15177 describes liquid crystal nicotine preparations to which a local anaesthetic is added to avoid disadvantageous effects of nicotine caused by its local irritant effect. In this case, the local anaesthetic acts by blocking peripheral pain receptors. It should be noted that cGMP PDE inhibitors on nasal administration cause such a local irritant effect to only a small extent or not at all.

GB-A-2 315 673 proposed intranasal administration of local anaesthetics such as lidocaine in addition to a 5-HT1D agonist for the treatment of migraines. Besides the effect of interrupting pain transmission which is known for local anaesthetics, this proposal is based on the vasodilating effect of local anaesthetics, which leads to an accelerated absorption of the 5-HT1D agonist and thus to a faster onset of action.

It would therefore have been expected that the disadvantages, described above, based on the vasodilating properties of cGMP PDE inhibitors would be further enhanced through the presence of a local anaesthetic because of its vasodilating effect.

It was the object of the present invention to find a composition for nasal administration of a cGMP PDE inhibitor, whose use is not associated with disadvantages such as a nasal condition which is found to be unpleasant, eye-watering, an increase in the nasal airway resistance or nasal blockage.

The above object is achieved by a composition which comprises at least one cGMP PDE inhibitor and at least one local anaesthetic, the local anaesthetic not being benzyl alcohol.

It has been found, surprisingly, that only a small amount of a local anaesthetic needs to be added to the compositions containing a cGMP PDE inhibitor, to overcome the disadvantages described above. The doses of local anaesthetic necessary for this purpose are generally distinctly less than those necessary for surface anaesthesia. A feeling of local numbness, as occurs after blockade of nerves conducting irritation, by, for example, a local anaesthetic, can therefore be avoided on use of the compositions according to the invention. Furthermore, addition of local anaesthetics to nasal compositions of cGMP PDE inhibitors surprisingly does not lead to build-up of excessive peaks in the plasma levels as would have been expected on the basis of the vasodilating properties of local anaesthetics and the accelerated and increased absorption of the cGMP PDE inhibitor in the nose which was thus to be expected. Thus, on use of the compositions according to the invention, no disadvantages in relation to the duration of action or increased side effects occur.

At present, 11 phosphodiesterases with varying specificity for the cyclic nucleotides cAMP and cGMP are described in the literature (cf. Fawcett et al., Proc. Natural. Acad. Sci. 97(7), 3072-3077 (2000)). Cyclic guanosine 3′, 5′-monophosphate-metabolizing phosphodiesterases (cGMP PDEs) are PDE-1, 2, 5, 6, 9, 10, 11. cGMP PDE inhibitors are thus, according to the present invention, compounds which inhibit one of more of these cGMP PDEs. Compositions preferred according to the invention are those which, besides one or more local anaesthetics, comprise one or more inhibitors of phosphodiesterase 5. A PDE 5 inhibitor is intended to mean, according to the present invention, a compound which chiefly inhibits PDE 5. Compositions preferred according to the invention are those which, besides one or more local anaesthetics, comprise one or more inhibitors of phosphodiesterase 5, which inhibits PDE 5 with an IC₅₀ value of less than 100 nM, preferably less than 30 nM, and has a selectivity for PDE 5 compared with PDE 1 by a factor of 50, preferably 100, and compared with PDE 4 by a factor of 300, preferably 1000. The IC₅₀ values can be determined for example by the procedure described in WO 99/24433. The contents of WO 99/24433 relating thereto is incorporated herein by reference. However, determination of the above IC₅₀ values is familiar to the skilled person in principle and can also be carried out in other ways.

According to a particularly preferred embodiment of the present invention, the cGMP PDE inhibitor is a compound of the formula (I)

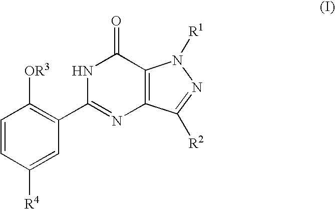

in which

R¹ is H; C₁-C₃-alkyl; C₁-C₃-perfluoroalkyl or C₃-C₅-cycloalkyl;

R² is H; optionally C₃-C₆-cycloalkyl-substituted C₁-C₆-alkyl, C₁-C₃-perfluoroalkyl or C₃-C₆-cycloalkyl;

R³ is optionally C₃-C₆-cycloalkyl-substituted C₁-C₆-alkyl; C₁-C₆-perfluoroalkyl; C₃-C₅-cycloalkyl; C₃-C₆-alkenyl or C₃-C₆-alkynyl;

R⁴ is C₁-C₄-alkyl which is optionally substituted by OH, NR⁵R⁶, CN, CONR⁵R⁶ or CO₂R⁷; C₂-C₄-alkenyl which is optionally substituted by CN, CONR⁵R⁶ or CO₂R⁷; C₂-C₄-alkanoyl which is optionally substituted by NR⁵R⁶; C₂-C₄-(hydroxy)alkyl which is optionally substituted by NR⁵R⁶; (C₂-C₃-alkoxy)-C₁-C₂-alkyl which is optionally substituted by OH or NR⁵R⁶; CONR⁵R⁶; CO₂R⁷; halogen; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl, each of which is optionally substituted by methyl;

R⁵ and R⁶ are each, independently of one another, H or C₁-C₄-alkyl or, together with the nitrogen atom to which they are bonded, form a pyrrolidinyl, piperidino, morpholino, 4-N(R¹¹)-piperazinyl or an imidazolyl group, this group optionally being substituted by methyl or OH;

R⁷ is H or C₁-C₄-alkyl;

R⁸ is optionally NR⁵R⁶-substituted C₁-C₃-alkyl;

R⁹ and R¹⁰ are, together with the nitrogen atom to which they are bonded, a pyrrolidinyl, piperidino, morpholino or 4-N(R¹²)-piperazinyl group, this group optionally being substituted by C₁-C₄-alkyl, C₁-C₃-alkoxy, NR¹³R¹⁴ or CONR¹³R¹⁴;

R¹¹ is H; optionally phenyl-substituted C₁-C₃-alkyl; (hydroxyl)-C₂-C₃-alkyl; or C₁-C₄-alkanoyl;

R¹² is H; C₁-C₆-alkyl; (C₁-C₃-alkoxy)-C₂-C₆-alkyl; (hydroxy)-C₂-C₆-alkyl; (R¹³R¹⁴N)C₂-C₆-alkyl; (R¹³R¹⁴NOC)-C₁-C₆-alkyl; CO—NR¹³R¹⁴; CSNR¹³R¹⁴ or C(NH)NR¹³R¹⁴; and

R¹³ and R¹⁴ are each, independently of one another, H; C₁-C₄-alkyl; (C₁-C₃-alkoxy)-C₂-C₄-alkyl or (hydroxy)-C₂-C₄-alkyl;

and salts, isomers and/or hydrates thereof.

According to a further preferred embodiment of the present invention, the cGMP PDE inhibitor is a compound of the formula (II)

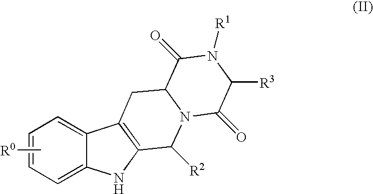

in which

R⁰ represents hydrogen, halogen or C₁₋₆-alkyl;

R¹ represents hydrogen, C₁₋₆-alkyl, C₂₋₆-alkenyl, C₂₋₆-alkynyl, halo-C₁₋₆-alkyl, C₃₋₈-cycloalkyl, C₃₋₈-cycloalkyl-C₁₋₃-alkyl, aryl-C₁₋₃-alkyl, where aryl is equal to phenyl or phenyl substituted by one to three substituents from the group consisting of halogen, C₁₋₆-alkyl, C₁₋₆-alkoxy, methylenedioxy and mixtures thereof, or represents heteroaryl-C₁₋₃-alkyl, where heteroaryl represents thienyl, furyl or pyridyl, each of which is optionally substituted by one to three substituents from the group consisting of halogen, C₁₋₆-alkyl, C₁₋₆-alkoxy, methylenedioxy and mixtures thereof;

R² represents an optionally substituted monocyclic aromatic ring selected from the group consisting of benzene, thiophene, furan and pyridine, or an optionally substituted bicyclic ring

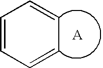

which is bonded to the remainder of the molecule via one of the carbon atoms of the benzene ring and in which the fused ring A is a 5- or 6-membered ring which may be saturated or partly or completely unsaturated and comprises carbon atoms and optionally one or two hetero atoms selected from the group consisting of oxygen, sulphur and nitrogen; and

R³ represents hydrogen or C₁₋₃-alkyl or

R¹ and R³ together represent a 3- or 4-membered alkyl or alkenyl chain moiety of a 5- or 6-membered ring,

and salts, isomers and/or hydrates thereof.

Compositions particularly preferred according to the invention contain as cGMP PDE inhibitor a compound selected from the group consisting of 1-{[3-(6,7-dihydro-1-methyl-7-oxo-3-propyl-1H-pyrazolo[4,3-d]-pyrimidin-5-yl)-4-ethoxyphenyl]sulphonyl}-4-methylpiperazine (Sildenafil) or (6R, 12aR)-2,3,6,7,12,12a-hexahydro-2-methyl-6-(3,4-methylenedioxyphenyl)pyrazino[2′,1′:6,1]pyrido[3,4-b]indole-1,4-dione, or their pharmaceutically acceptable salts, isomers and/or hydrates such as the corresponding hydrochloride, hydrochloride trihydrate, citrate or mesylate.

The compounds of the formula (I) can, for example, be prepared as described in EP-A-0 463 756 or EP-A-0 526 004. The compounds of the formula (H) can, for example, be prepared as described in WO 95/19978.

Unless otherwise indicated, the substituents generally have the following meaning for the purpose of the present invention:

Alkyl generally represents a straight-chain or branched hydrocarbon radical having 1 to 6 carbon atoms. Examples which may be mentioned are methyl, ethyl, propyl, isopropyl, butyl, isobutyl, pentyl, isopentyl, hexyl, isohexyl.

Alkenyl generally represents a straight-chain or branched hydrocarbon radical having 2 to 6 carbon atoms and one or more, preferably having one or two, double bonds. Examples which may be mentioned are allyl, propenyl, isopropenyl, butenyl, isobutenyl, pentenyl, isopentenyl, hexenyl and isohexenyl.

Alkynyl generally represents a straight-chain or branched hydrocarbon radical having 2 to 6 carbon atoms and one or more, preferably having one or two, triple bonds. Examples which may be mentioned are ethynyl, 2-butynyl, 2-pentynyl and 2-hexynyl.

Acyl generally represents straight-chain or branched lower alkyl having 1 to 6 carbon atoms which is linked via a carbonyl group. Examples which may be mentioned are: acetyl, ethylcarbonyl, propylcarbonyl, isopropylcarbonyl, butylcarbonyl and isobutylcarbonyl.

Alkoxy generally represents a straight-chain or branched hydrocarbon radical having 1 to 6 carbon atoms which is linked via an oxygen atom. Examples which may be mentioned are methoxy, ethoxy, propoxy, isopropoxy, butoxy, isobutoxy, pentoxy isopentoxy, hexoxy, isohexoxy. The terms “alkoxy” and “alkyloxy” are used synonymously.

Alkoxyalkyl generally represents an alkyl radical having up to 6 carbon atoms which is substituted by an alkoxy radical having up to 6 carbon atoms.

Alkoxycarbonyl can be represented, for example, by the formula

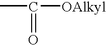

Alkyl in this case generally represents a straight-chain or branched hydrocarbon radical having 1 to 6 carbon atoms. Examples which may be mentioned are the following alkoxycarbonyl radicals: methoxycarbonyl, ethoxycarbonyl, propoxycarbonyl, isopropoxycarbonyl, butoxycarbonyl or isobutoxycarbonyl.

Cycloalkyl generally represents a cyclic hydrocarbon radical having 3 to 8 carbon atoms. Cyclopropyl, cyclopentyl and cyclohexyl are preferred. Examples which may be mentioned are cyclopentyl, cyclohexyl, cycloheptyl and cyclooctyl.

Halogen represents for the purpose of the invention fluorine, chlorine, bromine and iodine.

Heterocycle generally represents for the purpose of the invention a saturated, unsaturated or aromatic 3- to 6-membered, for example 5- or 6-membered, heterocycle which may contain up to 3 heteroatoms from the series S, N and/or O and, in the case of a nitrogen atom, may also be linked via the latter. Examples which may be mentioned are: oxadiazolyl, thiadiazolyl, pyrazolyl, pyridyl, pyrimidinyl, pyridazinyl, pyrazinyl, thienyl, furyl, pyrrolyl, pyrrolidinyl, piperazinyl, tetrahydropyranyl, tetrahydrofuranyl, 1,2,3 triazolyl, thiazolyl, oxazolyl, imidazolyl, morpholinyl or piperidyl. Thiazolyl, furyl, oxazolyl, pyrazolyl, triazolyl, pyridyl, pyrimidinyl, pyridazinyl and tetrahydropyranyl are preferred. The term “heteroaryl” (or “hetaryl”) represents an aromatic heterocyclic radical.

The above compounds of the formulae (I) and (II) may also be present in the form of their salts. Mention may be made here in general of salts with organic or inorganic bases or acids.

Physiologically acceptable salts are preferred for the purpose of the present invention. Physiologically acceptable salts of the compounds according to the invention may be salts of the substances according to the invention with mineral acids, carboxylic acids or sulphonic acids. Particularly preferred examples are salts with hydrochloric acid, hydrobromic acid, sulphuric acid, phosphoric acid, methanesulphonic acid, ethanesulphonic acid, p-toluenesulphonic acid, benzenesulphonic acid, naphthalenedisulphonic acid, acetic acid, propionic acid, lactic acid, tartaric acid, citric acid, fumaric acid, maleic acid or benzoic acid, and saccharic acids such as glucuronic acid or lactobionic acid.

Physiologically acceptable salts may likewise be metal or ammonium salts of the compounds according to the invention which have a free carboxyl group. Particularly preferred examples are sodium, potassium, magnesium or calcium salts, and ammonium salts which are derived from ammonia or organic amines such as, for example, ethylamine, di- or triethylamine, di- or triethanolamine, dicyclohexylamine, dimethylaminoethanol, arginine, lysine or ethylenediamine.

The compounds of the formulae (I) and (II) may exist in isomeric forms. This means according to the present invention stereoisomeric forms which are either related as image and mirror image (enantiomers) or not related as image and mirror image (diastereomers). The invention relates both to the enantiomers or diastereomers and to mixtures thereof in each case. The racemic forms may, just like the diastereomers, be separated in a known manner, for example by racemate resolution or chromatographic separation, into the stereoisomerically pure constituents. Double bonds present in the compounds according to the invention may be in the cis or trans configuration (Z or E form).

The compounds of the formulae (I) and (II) may also exist in the form of hydrates, in which case both hydrates of the free compounds and hydrates of salts thereof are encompassed by the present invention.

Compared with the amounts of cGMP PDE inhibitor required for oral administration, preferably amounts of only from 0.001 mg/kg to 0.5 mg/kg of cGMP PDE inhibitor are necessary with the compositions according to the invention which are to be administered nasally.

The local anaesthetics which can be used according to the invention are known per se and are listed, for example, in Remington's Pharmaceutical Sciences 1990, pp. 1048-1056. Local anaesthetics are compounds which reversibly inhibit the excitability of sensory nerve endings or the neuronal conductivity for pain or other sensory stimuli in a limited region of the body without causing permanent harm (cf. J. L. McGuire (editor), Pharmaceuticals, volume 2, Wiley-VCH, Weinheim 2000, pp. 539 et seq., Helwig/Otto, Arzneimittel [Medicinal products], volume II, Wissenschaftliche Verlagsgesellschaft mbH Stuttgart, 2000, pp. 37-1 et seq.). Local anaesthetics within the meaning of the present invention are preferably intended to mean substances which are listed in the Index Nominum 2000, International Drug Directory, Scientific Publishers Stuttgart 2000 with the therapeutic category “local anaesthetic”. Express reference is hereby made to the content concerning this in this reference.

Local anaesthetics preferred according to the present invention are compounds of the formula (III)

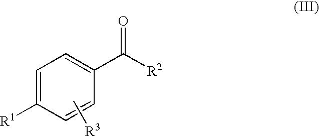

in which

R¹ represents H, NH₂, NH(C₁-6-alkyl), O—C₁-alkyl or CH₂OPh;

R² represents O—C₁₋₆-alkyl which may optionally have a radical from the group consisting of NH(C₁₋₆-alkyl), N(C₁₋₆-alkyl)₂ or a saturated 5- or six-membered heterocycle which contains at least one nitrogen atom and is linked via the latter, and optionally one or two further heteroatoms from the group consisting of N, O, S, and optionally carries one to three further C₁₋₆-alkyl radicals, or

represents (CH₂)₁₋₆-Het, where Het represents a saturated 5- or six-membered heterocycle which contains at least one nitrogen atom and is linked via the latter, and optionally one or two further heteroatoms from the group consisting of N, O, S, and optionally carries one to three further C₁₋₆-alkyl radicals;

R³ represents H, halogen or O—C₁₋₆-alkyl; or compounds of the formula (IV)

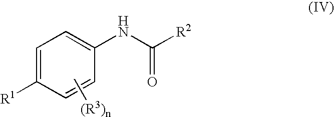

in which

R¹ represents H or OH;

R² represents C₁₋₆-alkyl-N(C₁₋₆-alkyl)₂ where the bridging alkyl chain may optionally carry one or more C₁₋₆-alkyl radicals, or represents a saturated 5- or six-membered heterocycle which contains at least one nitrogen atom and optionally one or two further heteroatoms from the group consisting of N, O, S, and optionally carries one to three further C₁₋₆-alkyl radicals,

R³ represents C₁₋₆-alkyl, halogen or COOC₁₋₆-alkyl;

n represents 1 or 2;

or a compound from the group consisting of

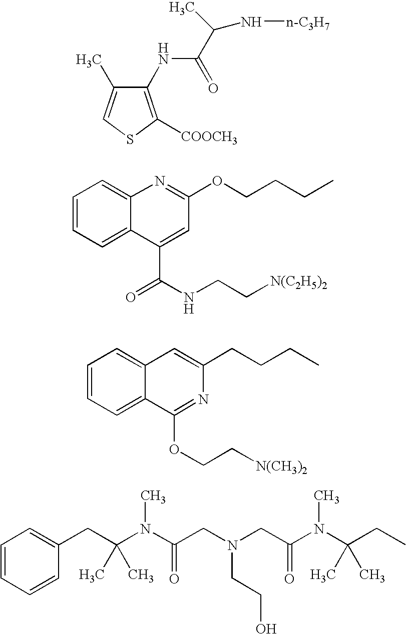

and polidocanol and benoxinate, and physiologically acceptable salts and/or hydrates thereof.

Particularly preferred local anaesthetics according to the invention are those of the formula (III)

in which

R¹ represents H, NH₂, NH-n-C₄H₉, O-n-C₃H₇, O-n-C₄H₉ or CH₂OPh;

R² represents OC₂H₅, O-n-C₄H₉, O-(CH₂)₂N(C₂H₅)₂, O(CH₂)₂N(CH₃)₂, or a radical from the group consisting of

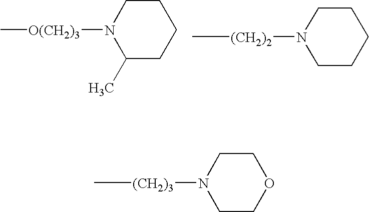

R³ represents H, Cl, O-n-C₃H₇ or O-n-C₄H₉;

or compounds of the formula (IV)

in which

R¹ represents H or OH;

R² represents CH₂N(C₂H₅)₂, CHCH₃NH-nC₃H₇, CH₂NH-n-C₄H₉ or a radical from the group consisting of

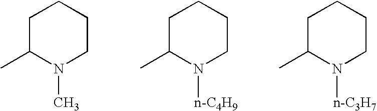

R³ represents CH₃, Cl or COOCH₃;

n represents 1 or 2;

and benoxinate and physiologically acceptable salts and/or hydrates thereof.

The local anaesthetics which can be particularly preferably employed according to the invention are: benzocaine, butambene, piperocaine, piperocaine hydrochloride, procaine, procaine hydrochloride, chloroprocaine, chloroprocaine hydrochloride, oxybuprocaine, oxybuprocaine hydrochloride, proxymetacaine, proxymetacaine hydrochloride, tetracaine, tetracaine hydrochloride, nirvanin, lidocaine, lidocaine hydrochloride, prilocaine, prilocaine hydrochloride, mepivacaine, mepivacaine hydrochloride, bupivacaine, bupivacaine hydrochloride, ropivacaine, ropivacaine hydrochloride, etidocaine, etidocaine hydrochloride, butanilicaine, butanilicaine hydrochloride, articaine, articaine hydrochloride, cinchocaine, cinchocaine hydrochloride, oxetacaine, oxetacaine hydrochloride, propipocaine, propipocaine hydrochloride, dyclonine, dyclonine hydrochloride, pramocaine, pramocaine hydrochloride, fomocaine, fomocaine hydrochloride, quinisocaine, quinisocaine hydrochloride, benoxinate and polidocanol. These compounds are commercially available or can be prepared in a way known to the skilled person, for example as described in J. L. McGuire (editor), Pharmaceuticals, volume 2, Wiley-VCH 2000, pp. 539 et seq.

Local anaesthetics which can preferably be used according to the invention are benzocaine, lidocaine, tetracaine, benoxinate, polidocanol or their pharmaceutically acceptable salts. Lidocaine hydrochloride and lidocaine methanesulphonate are particularly preferred according to the invention.

However, it should be pointed out once again that benzyl alcohol, which is occasionally referred to the local anaesthetic, is not encompassed by the present invention because it proved to be unsuitable for overcoming the disadvantages described above and, in addition, led to local irritation of the nasal mucosa.

The compositions according to the invention contain the local anaesthetic(s) in lower concentrations than the standard amount in commercially available topical preparations for surface anaesthesia, namely in a concentration of less than 4% (m/v), preferably less than 3% (m/v), where % (m/v) represents % mass/volume, that is to say 3% (m/v) means, for example, 3 g of substance in 100 ml of solution. According to the present invention, lidocaine is present in the compositions according to the invention in a concentration of less than 4% (m/v), preferably from 0.5 to 3.0% (m/v), which, with an administered volume of 100 μl corresponds to a single dose of less than 4 mg, preferably 0.5-3 mg. This is below the concentration of lidocaine in the commercial product Xylocain® 4%, which contains, for surface anaesthesia in the ear, nose and throat sector, 200 mg of lidocaine per 5 ml of volume (Rote Liste 1999, Editio Cantor, Aulendorf). According to the present invention, oxybuprocaine (benoxinate) is present in the compositions according to the invention in a concentration of less than 1% (m/v) (corresponding to a single dose of 0.5 mg/50 μl), preferably of 0.1-0.8% (m/v). For comparison, during surface anaesthesia in rhinology, a single dose of up to 105 mg of benoxinate per 70 kg of body weight is recommended (specialist information service Novesine® Wander 1%, 1998, quoted in: Drugdex Drug Evaluations, Micromedex 2001, Engelwood, Colo., USA). According to the present invention, tetracaine is present in the compositions according to the invention in a concentration of less than 0.5 mg per single dose, preferably of less than 0.25 mg per single dose. For comparison, up to 20 mg of tetracine is recommended for mucosal anaesthesia of the nose (Reynolds 1990, quoted in: Drugdex Drug Evaluations, Micromedex 2001, Engelwood, Colo., USA).

Intranasal preparations are known from the state of the art. The compositions according to the invention can be formulated analogously as solution, suspension, emulsion or powder for atomization in order to be sprayed, aspirated or introduced dropwise into the nose or applied to the mucous wall of the nose. Formulations in the form of a solution, suspension, for example a nanoparticle suspension, or emulsion can be administered as drop preparation for example from a nose drop bottle or a pipette, pump spray pack or compressed gas pack (for example an aerosol or an atomizing device), which can be calibrated in such a way that delivery of a fixed amount of the active ingredient(s) is possible. Powder preparations can be sprayed into the nose for example from a capsule provided with small perforations by means of a stream of air generated for example by a rubber bulb. All the preparation forms may represent multidose containers or divided single-dose containers.

Commercially available nasal applicators are, for example, the Pfeiffer unit dose and bidose system, the Valois monospray, bidose and monopowder system or the Becton-Dickinson Accuspray® system. Also suitable are glass or plastic bottles with commercially available metering pump spray heads.

Nanoparticle suspensions can be obtained by grinding powdered ingredients of the compositions according to the invention or by finely divided precipitation from solutions of ingredients of the formulations according to the invention and usually display improved solubility properties.

The compositions according to the invention contain, when formulated in liquid form, solvents and, where appropriate, one or more excipients such as, for example, buffers or substances for adjusting pH, viscosity-increasing substances, preservatives, surfactants, solubilizers, tonicity agents, antioxidants and flavourings.

Solvents which can be used according to the invention are water, glycerol, polyethylene glycol, propylene glycol or medium-chain triglycerides.

It is preferred according to the invention for liquid formulations of the compositions according to the invention to be adjusted to a pH in the range from 2 to 9, preferably 3 to 8, in order to avoid irritation in the nose and optimize the absorption of the cGMP PDE inhibitors. According to the present invention, this can be achieved by adding lactic acid (lactate), acetate, phosphate or citrate buffers or by adding methanesulphonic acid, hydrochloric acid, sulphuric acid, toluenesulphonic acid, gluconic acid, glucuronic acid, lactobionic acid, nitric acid, sodium hydroxide, potassium hydroxide, sodium carbonate or trometamol.

Viscosity-increasing excipients are, for example, polymers such as hydroxypropylmethylcellulose, hydroxypropylcellulose, methylcellulose, hydroxyethylcellulose, carboxymethylcellulose, carbomer, polyvinylpyrrolidone, polyvinyl alcohol or xanthan gum. Sugars or sugar alcohols such as sorbitol can also be used according to the present invention. The concentration of viscosity-increasing excipients in the compositions according to the invention can be chosen depending on the substance used and the required viscosity of the composition according to the invention.

The compositions according to the invention may furthermore contain one or more preservatives such as, for example, benzalkonium chloride, sorbic acid or its salts or benzoic acids or its salts, parabens such as methylparaben or propylparaben, chlorobutanol or thiomersal. The concentration of the preservative in the compositions according to the invention can be chosen depending on the substance used and the required application. A preservative if used is typically present in the compositions according to the invention in a concentration of up to 2% (m/v).

According to the present invention, the compositions according to the invention may also contain one or more surfactants and/or solubilizers in order, where appropriate, to increase the solubility of the cGMP PDE inhibitor used. It is possible to use for example according to the present invention polysorbates, polyethylene glycol, polyoxyethylene derivatives of fatty acid monoesters of sorbitol anhydrides such as, for example, Tweeen 80, polyoxyl 40 stearate, polyoxyethylene 50 stearate, bile salts, octoxynol, polyoxyethylated castor oil, polyoxystearate, poloxamers, phospholipid, benzoic acid, caffeine, vanilin, urea, nicotinamide, cyclodextrins or cyclodextrin ethers. It is possible according to the invention to use nonionic, anionic or cationic additives of the above categories. The concentration of the surfactants and/or solubilizers in the compositions according to the invention can be chosen depending on the substance used and the desired application. A surfactant and/or solubilizer if used is typically present in the compositions according to the invention in a concentration of from 0.001% (m/v) to about 5% (m/v).

According to the present invention, the compositions according to the invention may also contain one or more tonicity agents. Examples which can be used for this purpose according to the present invention are sodium chloride, calcium chloride, glycerol, mannitol or glucose. The concentration of the tonicity agents in the compositions according to the invention can be chosen depending on the substance used and the desired application. A tonicity agent if used is typically present in the compositions according to the invention in a concentration of from 0.001% (m/v) to about 5% (m/v).

According to the present invention, the compositions according to the invention may also contain one or more antioxidants. Examples which can be used for this purpose according to the present invention are sodium metabisulphite, sodium bisulphite, ascorbic acid and its salts, butylated hydroxytoluene, butylated hydroxyanisole, metal chelators such as ethylenediaminetetraacetic acid, butylated hydroxyanisole, propyl gallate, ascorbyl palmitate or tocopherol. The concentration of the antioxidants in the compositions according to the invention can be chosen depending on the substance used and the desired application. An antioxidant if used is typically present in the compositions according to the invention in a concentration of from 0.001% (m/v) to about 5% (m/v).

According to the present invention, the compositions according to the invention may also contain one or more flavourings. Examples which can be used for this purpose according to the present invention are saccharin sodium, aspartame, acesulphame potassium or menthol. The concentration of the flavourings in the compositions according to the invention can be chosen depending on the substance used and the desired application. A flavouring if used is typically present in the compositions according to the invention in a concentration of from 0.001% (m/v) to about 5% (m/v).

If the compositions according to the invention are administered in the from of compressed gas packs, these compressed gas packs additionally contain propellant gases such as, for example, propane, butane, nitrogen or nitrous oxide.

According to the present invention, compositions according to the invention in powder form additionally contain carriers such as, for example, glucose, sucrose, mannitol, crystalline cellulose or lactose.

According to the present invention, compositions according to the invention in powder form may also contain substances to prolong the contact time with the nasal mucosa such as, for example, polymers such as carbomer, chitosan or cellulose ethers. The concentration of these excipients in the compositions according to the invention can be chosen depending on the substance used and the desired application. Such an excipient is if used typically present in the compositions according to the invention in a concentration of from 0.001% (m/v) to about 5% (m/v).

According to the present invention, compositions according to the invention may additionally contain humectants in order to prevent or reduce drying out of the mucous membrane and thus prevent irritation. Examples which can be used for this purpose according to the present invention are sorbitol, propylene glycol or glycerol.

The concentration of the humectant in the compositions according to the invention can be chosen depending on the substance used and the desired application. A humectant is if used typically present in the compositions according to the invention in a concentration of from 0.001% (m/v) to about 5% (m/v).

The present invention is described in detail below by means of non-restrictive preferred examples. Unless otherwise indicated, all quantitative data relate to percentages by weight.

### EXAMPLES

Soluble formulations can be produced in a simple manner by dissolving the ingredients in the chosen solvent, then filtering the solution, charging the intended containers under aseptic conditions and, where appropriate, sterilizing with heat.

The cGMP PDE inhibitor can in this case be employed in the form of its salt chosen for the formulation. Alternatively, the free base can be added together with an appropriate acid to the above solution so that the corresponding salt is formed only in the solution. The subsequent further processing takes place in analogy to the procedure described above. It is thus possible for example to add the cGMP PDE inhibitor sildenafil in the form of its mesylate or as free base together with methanesulphonic acid to the above solution.

For administering higher doses and for avoiding stability problems, it may be advantageous to formulate the compositions according to the invention as powders. In this case, a particle size distribution of the powder formulation in the range from 1 to 100 μm, preferably from 5 to 40 μm, is desired because smaller particles may pass through the nose into the lungs, whereas larger particles are to some extent inadequately absorbed.

The appropriate containers for the finished formulations are known to the skilled person and are conventionally used single-dose or multidose containers.

Purified water means purified water as defined in the European Pharmacopoeia (Ph. Eur.) which is known to the skilled person. This is demineralized water of standardized quality.

### Example 1

A powder formulation is prepared from the following ingredients:

| | |
|---|---|
| Sildenafil citrate micronized | 25.0 kg |
| Lidocaine hydrochloride micronized | 10.0 kg |
| Lactose | 65.0 kg |

The ingredients are homogeneously mixed in an intensive mixer and packed in amounts of in each case 20 mg in single-dose powder insufflators.

### Example 2

A solution is prepared from the following ingredients:

| | |
|---|---|
| Sildenafil | 10.00 g |
| Lidocaine | 1.00 g |
| Lactic acid (20% solution) | 22.84 g |
| Purified water | 66.16 g |
| | 100.00 g |

The ingredients are dissolved in purified water, filtered and packed in 100 μl portions (+20 μl of non-removable overreach) in each case into the product container of a single-dose nasal applicator and heat sterilized. The product container is then incorporated into the single-dose nasal spray applicator. After actuation of the applicator in each case 100 μl of solution (corresponding to 10 mg of the cGMP PDE inhibitor employed) are delivered as aerosol.

1. Composition comprising at least one cGMP PDE inhibitor and at least one local anaesthetic, with the proviso that the local anaesthetic is not benzyl alcohol.

2. Composition according to claim 1, comprising a cGMP PDE inhibitor of the formula (I) 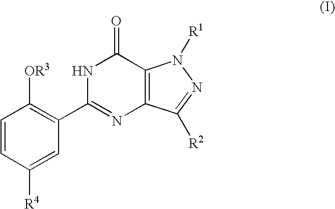
in which
R¹ is H; C₁-C₃-alkyl; C₁-C₃-perfluoroalkyl or C₃-C₅-cycloalkyl;
R² is H; optionally C₃-C₆-cycloalkyl-substituted C₁-C₆-alkyl, C₁-C₃-perfluoroalkyl or C₃-C₆-cycloalkyl;
R³ is optionally C₃-C₆-cycloalkyl-substituted C₁-C₆-alkyl; C₁-C₆-perfluoroalkyl; C₃-C₅-cycloalkyl; C₃-C₆-alkenyl or C₃-C₆-alkynyl;
R⁴ is C₁-C₄-alkyl which is optionally substituted by OH, NR⁵R⁶, CN, CONR⁵R⁶ or CO₂R⁷; C₂-C₄-alkenyl which is optionally substituted by CN, CONR⁵R⁶ or CO₂R⁷; C₂-C₄-alkanoyl which is optionally substituted by NR⁵R⁶; C₂-C₄-(hydroxy)alkyl which is optionally substituted by NR⁵R⁶; (C₂-C₃-alkoxy)-C₁-C₂-alkyl which is optionally substituted by OH or NR⁵R⁶; CONR⁵R⁶; CO₂R⁷; halogen; NR⁵R⁶; NHSO₂NR⁵R⁶; NHSO₂R⁸; SO₂NR⁹R¹⁰; or phenyl, pyridyl, pyrimidinyl, imidazolyl, oxazolyl, thiazolyl, thienyl or triazolyl, each of which is optionally substituted by methyl;
R⁵ and R⁶ are each, independently of one another, H or C₁-C₄-alkyl or, together with the nitrogen atom to which they are bonded, form a pyrrolidinyl, piperidino, morpholino, 4-N(R¹¹)-piperazinyl or an imidazolyl group, this group optionally being substituted by methyl or OH;
R⁷ is H or C₁-C₄-alkyl;
R⁸ is optionally NR⁵R⁶-substituted C₁-C₃-alkyl;
R⁹ and R¹⁰ are, together with the nitrogen atom to which they are bonded, a pyrrolidinyl, piperidino, morpholino or 4-N(R¹²)-piperazinyl group, this group optionally being substituted by C₁-C₄-alkyl, C₁-C₃-alkoxy, NR¹³R¹⁴ or CONR¹³R¹⁴;
R¹¹ is H; optionally phenyl-substituted C₁-C₃-alkyl; (hydroxyl)-C₂-C₃-alkyl; or C₁-C₄-alkanoyl;
R¹² is H; C₁-C₆-alkyl; (C₁-C₃-alkoxy)-C₂-C₆-alkyl; (hydroxy)-C₂-C₆-alkyl; (R¹³R¹⁴N)C₂-C₆-alkyl; (R¹³R¹⁴NOC)-C₁-C₆-alkyl; CO—NR¹³R¹⁴; CSNR¹³R¹⁴ or C(NH)NR¹³R¹⁴; and
R¹³ and R¹⁴ are each, independently of one another, H; C₁-C₄-alkyl; (C₁-C₃-alkoxy)-C₂-C₄-alkyl or (hydroxy)-C₂-C₄-alkyl; and salts, isomers and/or hydrates thereof.

3. Composition according to claim 2, comprising 1-{[3-(6,7-dihydro-1methyl-7-oxo-3-propyl-1H-pyrazolo[4,3-d]-pyrimidin-5-yl)-4-ethoxyphenyl]sulphonyl}-4-methylpiperazine or a salt, isomer and/or hydrate thereof as cGMP PDE inhibitor.

4. Composition according to claim 1, comprising a cGMP PDE inhibitor of the formula (II) 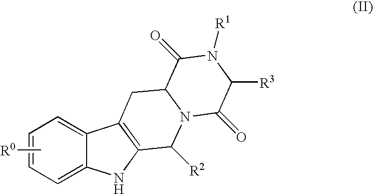
in which
R⁰ represents hydrogen, halogen or C₁₋₆-alkyl;
R¹ represents hydrogen, C₁₋₆-alkyl, C₂₋₆-alkenyl, C₂₋₆-alkynyl, halo-C₁₋₆-alkyl, C₃₋₈-cycloalkyl, C₃₋₈-cycloalkyl-C₁₋₃-alkyl, aryl-C₁₋₃-alkyl, where aryl is equal to phenyl or phenyl substituted by one to three substituents from the group consisting of halogen, C₁₋₆-alkyl, C₁₋₆-alkoxy, methylenedioxy and mixtures thereof, or represents heteroaryl-C₁₋₃-alkyl, where heteroaryl represents thienyl, furyl or pyridyl, each of which is optionally substituted by one to three substituents from the group consisting of halogen, C₁₋₆-alkyl, C₁₋₆-alkoxy, methylenedioxy and mixtures thereof;
R² represents an optionally substituted monocyclic aromatic ring selected from the group consisting of benzene, thiophene, furan and pyridine, or an optionally substituted bicyclic ring 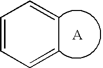
which is bonded to the remainder of the molecule via one of the carbon atoms of the benzene ring and in which the fused ring A is a 5- or 6-membered ring which may be saturated or partly or completely unsaturated and comprises carbon atoms and optionally one or two hetero atoms selected from the group consisting of oxygen, sulphur and nitrogen; and
R³ represents hydrogen or C₁₋₃-alkyl or
R¹ and R³ together represent a 3- or 4-membered alkyl or alkenyl chain moiety of a 5- or 6-membered ring,
and salts, isomers and/or hydrates thereof.

5. Composition according to claim 4, comprising (6R,12aR)-2,3,6,7,12,12a-hexahydro-2-methyl-6-(3,4-methylenedioxyphenyl)pyrazino[2′,1′:6,1]pyrido-[3,4-b]indole-1,4-dione or a salt, isomer and/or hydrate thereof as cGMP PDE inhibitor.

6. Composition according to any of claims 1 to 5, in which the local anaesthetic is selected from compounds of the formula (III) 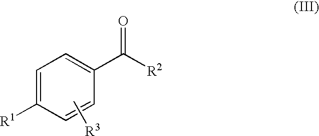
in which
R¹ represents H, NH₂, NH(C₁₋₆-alkyl), O—C₁₋₆-alkyl or CH₂OPh;
R² represents O—C₁₋₆-alkyl which may optionally have a radical from the group consisting of NH(C₁₋₆-alkyl), N(C₁₋₆-alkyl)₂ or a saturated 5- or six-membered heterocycle which contains at least one nitrogen atom and is linked via the latter, and optionally one or two further heteroatoms from the group consisting of N, O, S, and optionally carries one to three further C₁₋₆-alkyl radicals, or represents (CH₂)₁₋₆-Het, where Het represents a saturated 5- or six-membered heterocycle which contains at least one nitrogen atom and is linked via the latter, and optionally one or two further heteroatoms from the group consisting of N, O, S, and optionally carries one to three further C₁₋₆-alkyl radicals;
R³ represents H, halogen or O—C₁₋₆-alkyl;
or compounds of the formula (IV) 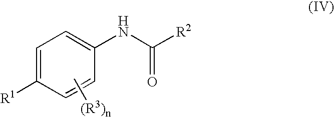
in which
R¹ represents H or OH;
R² represents C₁₋₆-alkyl-N(C₁₋₆-alkyl)₂ where the bridging alkyl chain may optionally carry one or more C₁₋₆-alkyl radicals, or represents a saturated 5- or six-membered heterocycle which contains at least one nitrogen atom and optionally one or two further heteroatoms from the group consisting of N, O, S, and optionally carries one to three further C₁₋₆-alkyl radicals,
R³ represents C₁₋₆-alkyl, halogen or COOC₁₋₆-alkyl;
n represents 1 or 2;
or a compound from the group consisting of 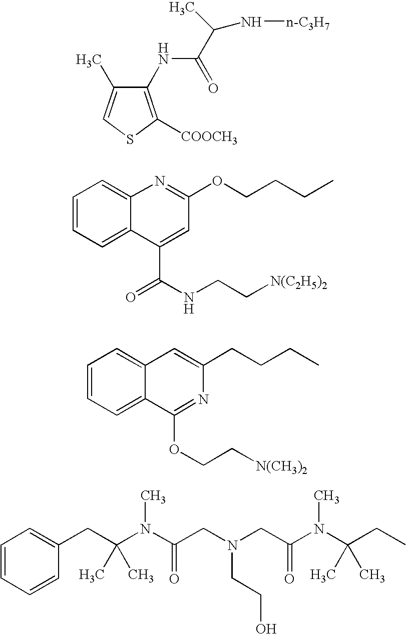
and polidocanol and benoxinate, and physiologically acceptable salts and/or hydrates thereof.

7. Composition according to claim 6, in which the local anaesthetic is selected from compounds of the formula (III)
in which
R¹ represents H, NH₂, NH-n-C₄H₉, O-n-C₃H₇, O-n-C₄H₉ or CH₂OPh;
R² represents OC₂H₅, O-n-C₄H₉, O-(CH₂)₂N(C₂H₅)₂, O(CH₂)₂N(CH₃)₂, or a radical from the group consisting of 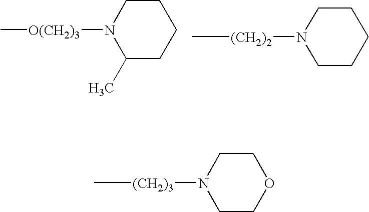
R³ represents H, Cl, O-n-C₃H₇ or O-n-C₄H₉;
or compounds of the formula (IV)
in which
R¹ represents H or OH;
R² represents CH₂N(C₂H₅)₂, CHCH₃NH-nC₃H₇, CH₂NH-n-C₄H₉ or a radical from the group consisting of 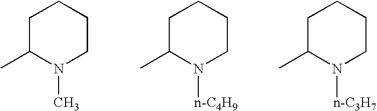
R³ represents CH₃, Cl or COOCH₃;
n represents 1 or 2;
and benoxinate and physiologically acceptable salts and/or hydrates thereof.

8. Composition according to claim 6, in which the local anaesthetic is selected from benzocaine, butambene, piperocaine, piperocaine hydrochloride, procaine, procaine hydrochloride, chloroprocaine, chloroprocaine hydrochloride, oxybuprocaine, oxybuprocaine hydrochloride, proxymetacaine, proxymetacaine hydrochloride, tetracaine, tetracaine hydrochloride, nirvanin, lidocaine, lidocaine hydrochloride, prilocaine, prilocaine hydrochloride, mepivacaine, mepivacaine hydrochloride, bupivacaine, bupivacaine hydrochloride, ropivacaine, ropivacaine hydrochloride, etidocaine, etidocaine hydrochloride, butanilicaine, butanilicaine hydrochloride, articaine, articaine hydrochloride, cinchocaine, cinchocaine hydrochloride, oxetacaine, oxetacaine hydrochloride, propipocaine, propipocaine hydrochloride, dyclonine, dyclonine hydrochloride, pramocaine, pramocaine hydrochloride, fomocaine, fomocaine hydrochloride, quinisocaine, quinisocaine hydrochloride, benoxinate and polidocanol.

9. Composition according to claim 6, in which the local anaesthetic is selected from the group consisting of benzocaine, lidocaine, tetracaine, benoxinate, polidocanol or their pharmaceutically acceptable salts.

10. Composition according to claim 6, where the local anaesthetic is lidocaine hydrochloride or lidocaine methanesulphonate.

11. Composition according to any of claims 1 to 10, where the local anaesthetic is present in a concentration of less than 4% (m/v).

12. Composition according to claim 11, where the local anaesthetic is present in a concentration of less than 3% (m/v).

13. Composition according to any of claims 1 to 12, where the cGMP PDE inhibitor is present in an amount of from 0.5 g/kg to 200 g/kg.

14. Composition according to any of claims 1 to 13, additionally comprising solvents and one or more excipients from the group consisting of buffers or substances to adjust the pH, viscosity-increasing substances, preservatives, surfactants, solubilizers, tonicity agents, antioxidants, flavourings, substances to prolong the contact time and humectants.

15. Composition according to any of claims 1 to 14, further comprising one or more excipients from the group consisting of buffers or substances to adjust the pH, viscosity-increasing substances, preservatives, surfactants, solubilizers, tonicity agents, antioxidants, flavourings, carriers, substances to prolong the contact time and humectants.

16. Composition according to any of claims 1 to 15 for treating diseases.

17. Pharmaceutical composition for nasal administration, comprising a composition according to any of claims 1 to 16.

18. Use of a composition according to any of claims 1 to 17 for producing a medicinal product for treating male erectile dysfunction.

19. Use according to claim 18, where the treatment takes place by nasal administration.

20. Nasal spray applicator comprising a composition according to any of claims 1 to 17.

21. Nasal spray applicator according to claim 20, which is a single-dose nasal spray applicator.

22. Powder insufflator comprising a composition according to any of claims 1 to 17.

23. Powder insufflator according to claim 22, which is a single-dose powder insufflator.
